(19) United States
(12) Patent Application Publication
(10) Pub. No.: US 2024/0116055 A1
(43) Pub. Date: Apr. 11, 2024

(54) DIGITAL MICROFLUIDIC DEVICES WITH PARKING ELECTRODES

(71) Applicant: Hewlett-Packard Development Company, L.P., Spring, TX (US)

(72) Inventors: Michael W. Cumbie, Corvallis, OR (US); Chien-Hua Chen, Corvallis, OR (US); Viktor Shkolnikov, Palo Alto, CA (US)

(73) Assignee: Hewlett-Packard Development Company, L.P., Spring, TX (US)

(21) Appl. No.: 17/960,942

(22) Filed: Oct. 6, 2022

(51) Int. Cl. B01L 3/00; C12M 3/06

(52) U.S. Cl. CPC B01L 3/502792; B01L 3/502715; B01L 3/50273; C12M 23/16; B01L 2200/027; B01L 2300/0645; B01L 2300/0816; B01L 2300/161; B01L 2300/165; B01L 2400/0427

(57) ABSTRACT

An example digital microfluidic device can include a hydrophobic electrowetting surface including an array of electrodes. The individual electrodes can have a shape with three or more sides. The array of electrodes can include a parking electrode and an adjacent electrode that is adjacent to the parking electrode. A cover can be positioned over the electrowetting surface at a gap distance sufficient to accommodate a liquid droplet between the cover and the electrowetting surface. A plurality of droplet barriers can be positioned on three or more sides of the parking electrode. The droplet barriers can constrain movement of a liquid droplet from the parking electrode to the adjacent electrode.

### BACKGROUND

Microfluidics relates to the behavior, precise control and manipulation of fluids in small quantities, such as milliliters, microliters, nanoliters, or smaller volumes. Digital microfluidics, in particular, can relate to control and movement of discrete volumes of fluids. A variety of applications for microfluidics exists with various applications involving differing controls over fluid flow, mixing, temperature, evaporation, and so on.

### BRIEF DESCRIPTION OF THE DRAWINGS

FIG. 1 is a top plan view of an example digital microfluidic device in accordance with the present disclosure:

FIGS. 2A-2C are top plan views of an example digital microfluidic device moving a liquid droplet in accordance with the present disclosure;

FIGS. 3A-3C are side views of an example digital microfluidic device moving a liquid droplet in accordance with the present disclosure;

FIG. 4 is a side view of another example digital microfluidic device in accordance with the present disclosure;

FIG. 5 is a side view of another example digital microfluidic device in accordance with the present disclosure:

FIG. 6 is a side view of another example digital microfluidic device in accordance with the present disclosure;

FIG. 7 is a side view of still another example digital microfluidic device in accordance with the present disclosure;

FIG. 8 is a side view of another example digital microfluidic device in accordance with the present disclosure:

FIG. 9 is a side view of another example digital microfluidic device in accordance with the present disclosure;

FIG. 10 is a top plan view illustrating several designs for droplet barriers in accordance with the present disclosure;

FIG. 11 is a top plan view of another example digital microfluidic device in accordance with the present disclosure;

FIG. 12 is a flowchart illustrating an example method of making a digital microfluidic device in accordance with the present disclosure;

FIGS. 13A-13B are side views illustrating an example method of making a digital microfluidic device in accordance with the present disclosure;

FIGS. 14A-14B are side views illustrating another example method of making a digital microfluidic device in accordance with the present disclosure;

FIGS. 15A-15B are side views illustrating another example method of making a digital microfluidic device in accordance with the present disclosure; and

FIG. 16 is a flowchart illustrating an example method of processing a liquid in a digital microfluidic device in accordance with the present disclosure.

### DETAILED DESCRIPTION

The present disclosure describes digital microfluidic devices with an electrowetting surface and parking electrodes for holding droplets in place over periods of time, even when power to the electrodes is turned off. The digital microfluidic devices can include an array of electrodes that can move droplets of liquid across the array using electrowetting forces. These devices may be used for a variety of applications, such as mixing droplets of multiple reactants, splitting droplets, incubating cells, DNA amplification, and others. Electrowetting forces can hold a droplet in place over an electrode while a voltage is applied to that electrode. However, if voltage to the electrode array is turned off for a period of time then the droplet may drift randomly to other locations on the electrowetting surface. Certain processes may call for the digital microfluidic device to be disconnected from electrical power for a period of time. For example, the device may be disconnected from power and placed in an oven for incubation of cells or thermal cycling in a DNA amplification process. In another example, the device may be disconnected from power and loaded in a detection instrument for data analysis. In such processes, undesired drifting of droplets can occur while the power is disconnected, and this can lead to poor results. To prevent unwanted drifting of droplets, the digital microfluidic devices described herein include parking electrodes with droplet barriers that can constrain movement of droplets off of the parking electrodes. The droplet barriers can be designed to allow for intentional movement of droplets on and off of the parking electrodes through the application of voltage to the electrode array, but to prevent undesired drifting of the droplets when the voltage is turned off.

In one example, a digital microfluidic device includes a hydrophobic electrowetting surface having an array of electrodes. The individual electrodes have a shape with three or more sides. The array of electrodes includes a parking electrode and an adjacent electrode that is adjacent to the parking electrode. A cover is positioned over the electrowetting surface at a gap distance sufficient to accommodate a liquid droplet between the cover and the electrowetting surface. A plurality of droplet barriers is positioned on three or more sides of the parking electrode. The droplet barriers constrain movement of a liquid droplet from the parking electrode to the adjacent electrode. In certain examples, the droplet barriers can have a surface having a greater hydrophobicity compared to the parking electrode. In further examples, the droplet barriers can include solid walls occupying a space between the cover and the electrowetting surface. The solid walls can extend across the entire gap distance between the electrowetting surface and the cover. Alternatively, the solid walls can extend partially across the distance between the electrowetting surface and the cover. The solid walls can be formed as a part of the electrowetting surface or as a part of the cover. In other examples, the droplet barriers can include grooves formed in the electrowetting surface. The digital microfluidic device can also include an oil filling a volume between the cover and the electrowetting surface. The device can also include a droplet of an aqueous liquid positioned over the parking electrode.

The present disclosure also describes methods of making digital microfluidic devices. In one example, a method of making a digital microfluidic device includes placing a cover over a hydrophobic electrowetting surface at a gap distance sufficient to accommodate a liquid droplet between the cover and the electrowetting surface. The electrowetting surface includes an array of electrodes. The individual electrodes have a shape with three or more sides. The array of electrodes includes a parking electrode and an adjacent electrode that is adjacent to the parking electrode. The method further includes forming a plurality of droplet barriers positioned on three or more sides of the parking electrode. The droplet barriers constrain movement of a liquid droplet from the parking electrode to the adjacent electrode. In certain examples, forming the plurality of droplet barriers can include forming solid walls extending from the electrowetting surface or extending from the cover. The solid walls can be formed by molding, machining, photoresist patterning, embossing, stamping, or a combination thereof. In further examples, forming the plurality of droplet barriers can include forming a surface having a greater hydrophobicity compared to the parking electrode. The surface having a greater hydrophobicity can be formed by stamping a hydrophobic coating composition, selectively dispensing a hydrophobic coating composition, selective surface modification using a stencil, forming a hydrophobic nanostructure, laser etching, reactive ion etching, or a combination thereof.

The present disclosure also describes methods of processing liquids using digital microfluidic devices. In one example, a method of processing a liquid in a digital microfluidic device includes moving an aqueous liquid droplet by applying voltage to electrodes on a hydrophobic electrowetting surface of a digital microfluidic device. The electrowetting surface includes an array of electrodes, where the array of electrodes includes a parking electrode and an adjacent electrode that is adjacent to the parking electrode. While the aqueous liquid droplet is positioned over the parking electrode, the voltage is turned off to the electrode array for a period of time. The position of the aqueous liquid droplet is maintained over the parking electrode during the period of time using a plurality of droplet barriers positioned on sides of the parking electrode. In further examples, the method can include, during the period of time while the voltage is turned off, storing the digital microfluidic device, shipping the digital microfluidic device, loading the digital microfluidic device into an oven, applying heat to the digital microfluidic device, applying thermal cycling to the digital microfluidic device, loading the digital microfluidic device into an instrument for data analysis, or a combination thereof. In certain examples, the digital microfluidic device can include a cover positioned over the electrowetting surface at a gap distance sufficient to accommodate the aqueous liquid droplet between the cover and the electrowetting surface, and the droplet barriers can include a surface having a greater hydrophobicity compared to the parking electrode, or solid walls occupying a space between the cover and the electrowetting surface, or a combination thereof.

The digital microfluidic devices described herein can utilize electrowetting forces to control the movement of droplets of liquid on an electrowetting surface. Electrowetting refers to a change in contact angle between a liquid and a solid surface when an electric field is applied between the liquid and the solid surface. In some cases, an electrowetting surface can include a relatively hydrophobic surface that is in contact with the liquid droplet. Thus, the surface can have a relatively large contact angle with the liquid droplet, such as greater than 90° in some examples. However, applying an electric field can effectively make the surface more wettable. In other words, the surface and the liquid droplet can behave as if the surface is more hydrophilic when the electric field is applied. This effect can be due to a combination of forces including surface tension and electric forces.

The electrowetting effect can be used, in some examples, to cause liquid droplets to move across the electrowetting surface. For example, an electric field can be applied to an area of the surface near or adjacent to the location of a liquid droplet. The liquid can have a smaller contact angle with the surface in the area of the electric field than in the area outside the electric field. This can cause the liquid to preferentially wet the surface in the adjacent area where the electric field is applied. Thus, the liquid droplet can physically move into the area where the electric field is applied as the liquid wets the surface in this area, while leaving the more hydrophobic area of the surface outside the electric field.

Such surfaces can be included in digital microfluidic devices. Digital microfluidic devices can be designed in a variety of ways. In many examples, digital microfluidic devices can be capable of moving multiple discrete droplets of liquid across their electrowetting surfaces. In some cases, the movement of many droplets can be controlled independently, which can allow the individual droplets to be directed to locations, combined with other droplets, split to form smaller droplets, and so on. Some digital microfluidic devices include an array of electrodes located under a layer of dielectric material. A voltage can be applied to an individual electrode to cause a liquid droplet to move to the surface over the individual electrode. By individually controlling the voltage of the electrodes in the array, such devices can control the movement of multiple liquid droplets across the hydrophobic surface. These devices can be used for a variety of applications, such as dividing a quantity of liquid into multiple droplets having a known volume, or separating specific species from other species in a liquid, or combining droplets containing different reactants to cause chemical reactions, or other applications.

Maintaining a voltage applied to one electrode on an electrowetting surface can effectively hold a liquid droplet in place over that electrode. The voltage creates the electrowetting effect that makes the surface more wettable over the specific electrode that has a voltage applied. Therefore, the liquid droplet does not move away from the electrode because the surface around the electrode is less wettable. However, this effect can disappear if the voltage to the electrode is turned off. If the voltage is turned off to all the electrodes on the electrowetting surface, then the liquid droplet can tend to drift in random directions across the surface. This is because there is no location that is energetically more favorable for the droplet to stay in place. Thus, with many electrowetting devices, turning off the voltage to the electrodes creates a risk that liquid droplets will wander at random on the surface.

There are multiple situations in which it can be useful to turn off voltage to electrodes of a digital microfluidic device for a period of time. For example, a digital microfluidic device may be used to process droplets of liquids and then the device may be disconnected from power and then loaded into an oven for a thermal treatment such as incubation or thermal cycling. In another example, a digital microfluidic device may be used to process droplets of liquids and then may be disconnected from power and loaded into a separate detection instrument for data analysis. In such processes, the voltage used to control the droplets is no longer present when the device is disconnected from power. Therefore, the droplets can tend to drift in a random, uncontrolled manner across the electrowetting surface of the device.

The digital microfluidic devices described herein can prevent undesired drifting of droplets, even when the devices are disconnected from electrical power. The devices include parking electrodes that are designed to hold droplets in place even when no voltage is applied to the electrode. This can be accomplished by including droplet barriers positioned around the parking electrode or between the parking electrode and adjacent electrodes to constrain movement of a droplet from the parking electrode to the adjacent electrode. The droplet barriers can include physical walls that physically constrain the droplet, or features that have increased hydrophobicity (i.e., lower surface energy), or combinations thereof. Thus, when voltage to the parking electrode is turned off, the droplet can still stay in place due to the droplet barriers. Various types and arrangements of droplet barriers are described in more detail below.

As used herein, “constrain movement” means that the droplet barriers discourage the movement of the droplet due to random drifting when the electrodes are unpowered. However, the droplet barriers can be designed to allow the droplet to move when desired, by applying voltage to an adjacent electrode of the electrowetting surface. Thus, the droplet barriers may be designed so that the parking electrode is not completely blocked off from adjacent electrodes, but a pathway can exist for the droplet to move when acted upon by electrowetting forces. In some examples, a relatively higher voltage may be used to move droplets onto and off of a parking electrode because of the resistance introduced by the droplet barriers. The normal voltage used to move droplets between normal (i.e., non-parking) electrodes may not be sufficient to move a droplet onto or off of the parking electrode. However, the droplet barriers can be designed so that a suitable voltage can be applied to the parking electrode to move a droplet onto the parking electrode, and likewise a suitable voltage can be applied to an adjacent electrode to move the droplet off of the parking electrode.

It is noted that liquid droplets on the electrowetting surface are usually not in direct contact with the electrodes. The electrowetting surface of a digital microfluidic device can include the array of electrodes and a layer of dielectric material over the electrodes. Additional layers may also be added, such as a hydrophobic layer over the dielectric layer. Thus, the liquid droplets on the electrowetting surface do not directly contact the electrodes. As used herein, stating that a droplet is “on” an electrode can mean the same thing as stating that the droplet is “over” the electrode, which can mean that the droplet is in contact with the dielectric layer or hydrophobic layer laying over the electrode.

As used herein, “hydrophobic surface” can refer to a surface that has a contact angle greater than 90° with water. Additionally, “more hydrophobic” and “increased hydrophobicity” can mean a surface that has a comparatively greater contact angle with water.

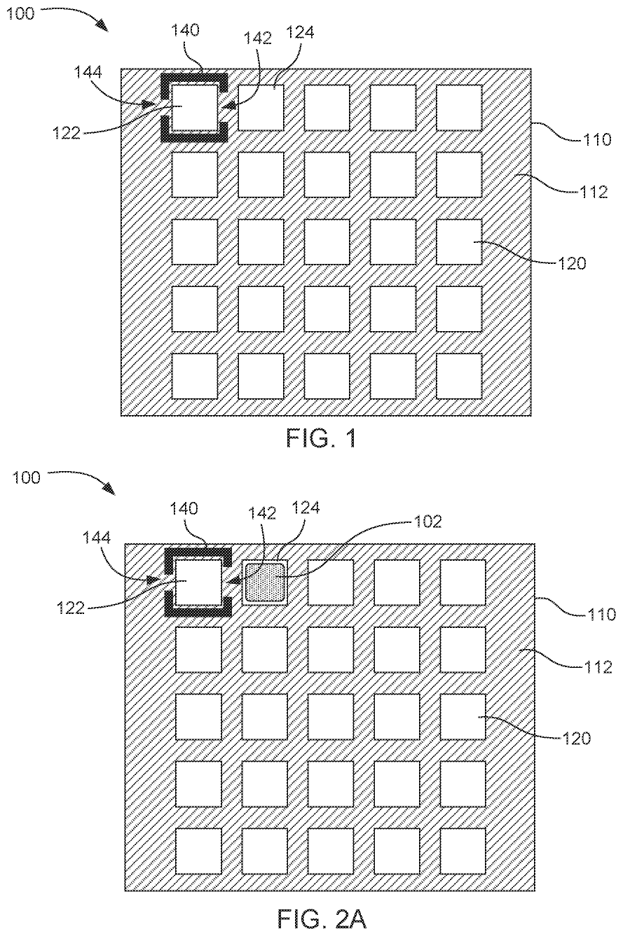

An example digital microfluidic device 100 is shown in FIG. 1. The device includes a hydrophobic electrowetting surface 110 including a substrate 112 and an array of electrodes 120 on the substrate. The individual electrodes have a shape with three or more sides. In this particular example, the electrodes have a square shape. The array of electrodes includes a parking electrode 122 and an adjacent electrode 124 that is adjacent to the parking electrode. The device can also include a dielectric layer over the electrodes and the substrate, but this dielectric layer is not shown in this image so that the electrodes can be clearly visible. A cover is positioned over the electrowetting surface at a gap distance sufficient to accommodate a liquid droplet between the cover and the electrowetting surface. However, the cover is not shown in this figure. The cover can be transparent in some examples, so that the electrodes and droplets moving on the electrodes can be visible. A plurality of droplet barriers 140 are positioned on sides of the parking electrode. The droplet barriers are designed to constrain movement of a liquid droplet from the parking electrode. In this example, the droplet barriers include walls that partially encompass four sides of the square parking electrode. Thus, in this example the barriers are positioned on four sides of the parking electrode. The walls are formed such that a droplet entrance 142 is present between the parking electrode and the adjacent electrode. This droplet entrance allows a droplet to move from the adjacent electrode to the parking electrode and vice versa. The gap between the electrowetting surface and the cover can often be filled with air or an oil, and droplets of aqueous liquid can move through the air or oil by electrowetting forces. When the parking electrode is partially enclosed by walls as in this example, it can be helpful to provide an escape for the air or silicon oil that is displaced when a droplet of aqueous liquid moves onto the parking electrode. Accordingly, this example includes an exit port 144 formed as an opening in the walls on the side of the parking electrode. When a droplet of aqueous liquid moves from the adjacent electrode onto the parking electrode, the air or oil that is displaced by the droplet can exit through this exit port.

The example shown in FIG. 1 includes a parking electrode with droplet barriers that are positioned on four sides of the parking electrode. The droplet barriers do not completely encompass all four sides, but a portion of all four sides is encompassed so that a liquid droplet positioned on the parking electrode is constrained no matter which direction the droplet might drift. In various examples, the digital microfluidic devices described herein can include droplet barriers positioned on three or more sides of the parking electrode. If the droplet barriers are positioned on less than three sides, then the liquid droplet may not be sufficiently contained to prevent drifting. For example, if a square parking electrode has a barrier on two sides, then the other two sides are left open to allow the droplet to drift without being constrained.

It is noted that the electrodes and the droplet barriers can have a variety of shapes and arrangements. In some examples, the electrodes can have a square shape as in the example of FIG. 1. In other examples, the electrodes can be shaped as another polygon such as a triangle, rectangle, pentagon, hexagon, and so on. In such examples, the droplet barriers can be positioned on three or more sides of the electrodes, referring to three or more sides of the polygon shape of the electrodes. In certain examples, the electrodes can have a rounded or circular shape. In other examples, the droplet barriers can have a rounded or circular shape. In such examples, “three or more sides” can mean that a droplet barrier encompasses more than 180° of the circular electrode or circular shape of the droplet barrier. The droplet barriers can include gaps, such as the droplet entrance and exit port in the example of FIG. 1. The droplet barriers can also be described by how they constrain the movement of a liquid droplet. When droplet barriers are positioned on three sides of the parking electrode, then a liquid droplet on the parking electrode is constrained when moving in all directions except one. The single unconstrained direction is the direction toward the side that does not have a droplet barrier. When droplet barriers are positioned on four sides of the parking electrode, then the droplet is constrained when moving in any direction. As used herein, “constrained” means that the droplet barriers either completely physically block movement, or if there is a gap in the droplet barrier then the gap is smaller than the width of the droplet so that the droplet cannot move through the gap without deformation. In certain examples, the droplet barriers can be designed to constrain movement of the droplet in all directions in this way.

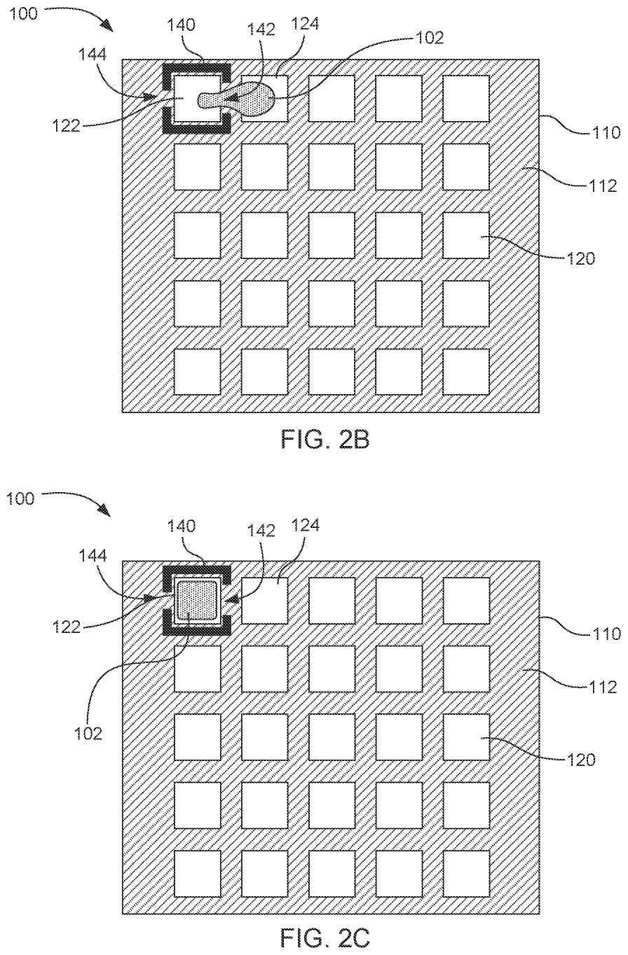

An example of a liquid droplet moving from an adjacent electrode to a parking electrode is shown in FIGS. 2A-2C. FIG. 2A shows a digital microfluidic device 100 that includes a hydrophobic electrowetting surface 110 including a substrate 112 and an array of electrodes 120. A dielectric layer is positioned over the electrodes and the substrate, but again the dielectric layer is not shown so that the electrodes can be clearly visible in the figure. The electrodes include a parking electrode 122 and an adjacent electrode 124 adjacent to the parking electrode. Droplet barriers 140 are positioned on four sides of the parking electrode, with openings for a droplet entrance 142 on the right side and an exit port 144 on the left side. In this figure, a liquid droplet 102 is positioned on the adjacent electrode. The liquid droplet can be held in place on the adjacent electrode by applying a voltage to the adjacent electrode. FIG. 2B shows the droplet beginning to move toward the parking electrode. This can occur because the voltage to the adjacent electrode is discontinued and a voltage is applied to the parking electrode. The voltage can cause the surface over the parking electrode to be more wettable, which can attract the droplet of aqueous liquid. This figure shows the droplet stretching toward the parking electrode and squishing through the droplet entrance opening in the droplet barrier walls. FIG. 2C shows the droplet after it has moved fully onto the parking electrode. In this position the droplet can be held in place by applying a voltage to the parking electrode. However, the droplet can also remain in place even if the voltage is turned off to the parking electrode because the droplet barriers constrain the motion of the droplet. This can prevent the droplet from drifting away when the voltage is turned off. It is noted that when it is desired to move the droplet off the parking electrode and onto the adjacent electrode, the process shown in FIGS. 2A-2C can be reversed simply by applying a voltage to the adjacent electrode.

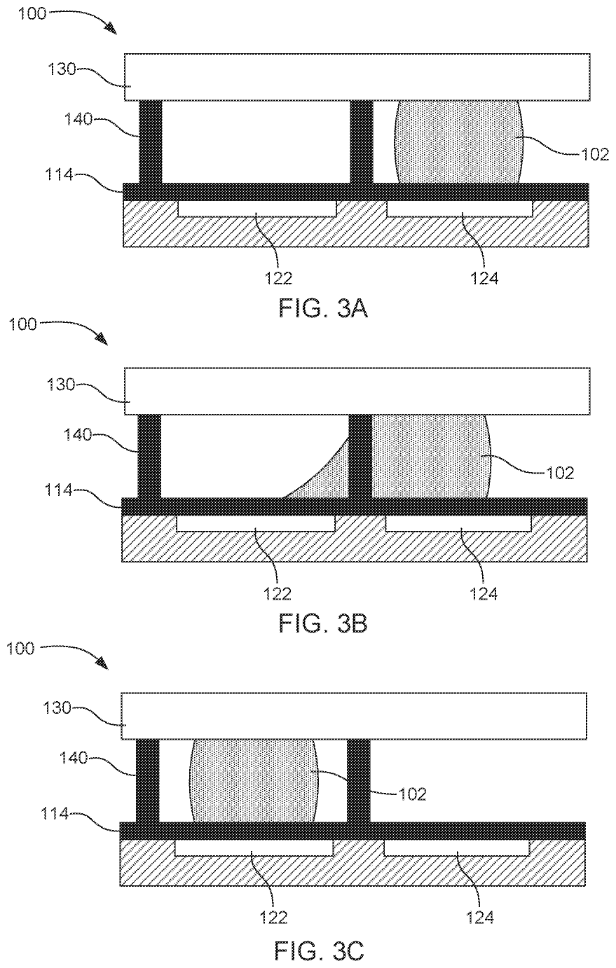

FIGS. 3A-3C show a side view of the same process of a liquid droplet 102 moving from an adjacent electrode 124 to a parking electrode 122. The side view shows the hydrophobic electrowetting surface 110 including a substrate 112 and electrodes 120 on the substrate. These figures do not show the entire array of electrodes, but show a close-up view of the parking electrode and the adjacent electrode. A dielectric layer 114 is positioned over the electrodes and the substrate. Droplet barriers 140 are shown positioned on sides of the parking electrode. In this example, the droplet barriers are walls formed in the dielectric layer material. In other examples, the droplet barriers can be formed of a different material that is deposited onto the dielectric layer, or the droplet barriers can be formed of the substrate material, or a separate material that is deposited on the substrate layer. A cover 130 is positioned over the electrowetting surface at a gap distance that allows the liquid droplet to be accommodated between the cover and the electrowetting surface. In this example, the droplet barriers extend across the entire gap between the electrowetting surface and the cover. FIG. 3A shows the liquid droplet on the adjacent electrode. FIG. 3B shows the droplet as it begins to move toward the parking electrode. As explained above, this can be accomplished by applying a voltage to the parking electrode. This results in a decrease in the wetting angle between the liquid droplet and the surface over the parking electrode. FIG. 3C shows the liquid droplet after moving onto the parking electrode. The liquid droplet is held in place by the droplet barriers, which prevent the droplet from drifting off of the parking electrode when the voltage to the parking electrode is turned off.

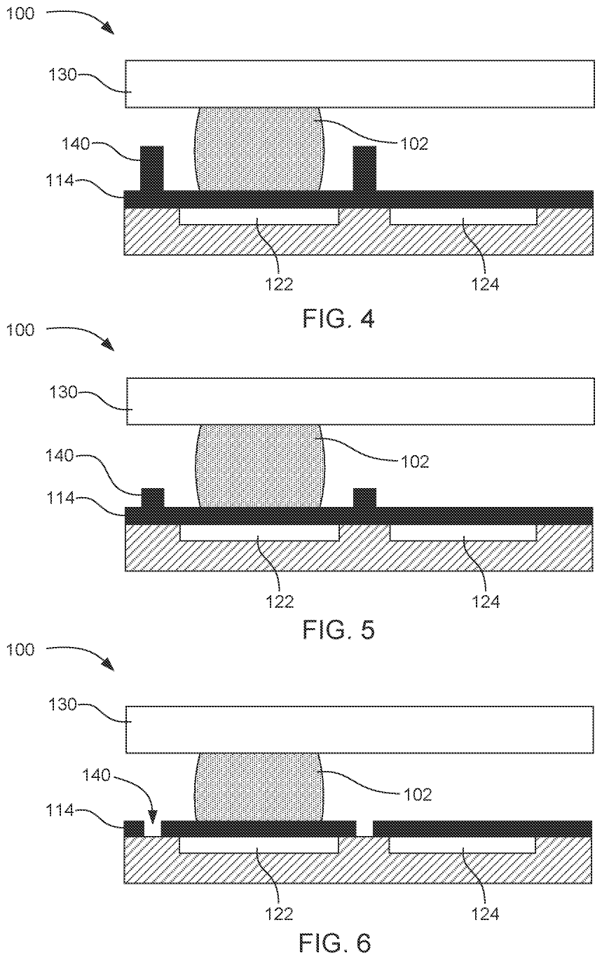

In the example shown in FIGS. 3A-3C, the droplet barriers are walls that extend all the way from the electrowetting surface to the cover. Thus, the walls do not allow liquid to move over or under the walls. Therefore, the walls can be designed to include gaps for a droplet entrance and an exit port as explained above. In other examples, the droplet barriers can include walls that extend partially across the gap between the electrowetting surface and the cover. FIG. 4 shows a side view of an example digital microfluidic device 100 that has droplet barriers 140 formed as walls that extend from the electrowetting surface 110 about halfway across the gap distance to the cover 130. With this design, it is possible for the liquid droplet 102 to move through the space between the walls and the cover. However, the walls can be sufficient to prevent random drifting of the liquid droplet when voltage to the parking electrode 122 is turned off.

FIG. 5 shows a cross-sectional view of another example digital microfluidic device 100. This example includes droplet barriers 140 that are short walls, which extend less than half way across the gap distance between the electrowetting surface 110 and the cover 130. These walls can also be sufficient to hold the liquid droplet 102 in place and prevent drifting when the voltage to the parking electrode 122 is turned off.

FIG. 6 shows another side view of an example digital microfluidic device 100. This example includes a different type of droplet barriers 140. These droplet barriers are formed as grooves in the electrowetting surface instead of as walls. The grooves can prevent drifting of the liquid droplet 102 because the surface tension of the liquid and the hydrophobic nature of the surface make it energetically unfavorable for the liquid to flow into the groove.

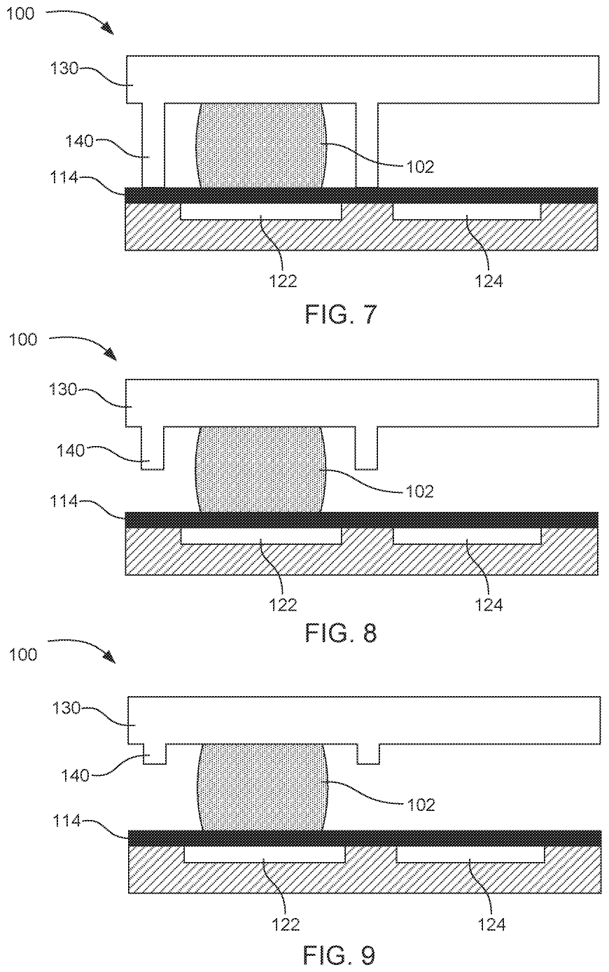

The droplet barriers can also be formed on the cover instead of on the electrowetting surface. Any of the types of droplet barriers described herein can be on either the electrowetting surface or the cover, in particular the inside surface of the cover that contacts the liquid droplets. FIG. 7 is a side view of an example digital microfluidic device 100 where droplet barriers 140 are formed as walls extending down from the cover to the electrowetting surface. These walls can have a shape similar to that shown in FIG. 1, as viewed from above (i.e., looking down on the cover of the device). The walls can include a droplet entrance and an exit port, although these features are not visible in FIG. 7.

FIG. 8 shows a side view of another example digital microfluidic device 100 that includes droplet barriers 140 formed as walls that extend from the cover about half way down to the electrowetting surface. As explained above, it is possible for the liquid droplet to move under these droplet barriers, if a sufficient force is applied, but the barriers can be sufficient to prevent random drifting of the droplet when the voltage is turned off to the parking electrode 122.

FIG. 9 shows a side view of another example digital microfluidic device 100. This example includes droplet barriers 140 that are formed as short walls that extend less than half way down to the electrowetting surface. These can also be sufficient to prevent random drifting of the droplet when the voltage is turned off to the parking electrode 122.

The dimensions of the droplet barriers can vary widely, as the size of digital microfluidic devices can vary from one device to the next. In some examples, droplet barriers can have a width from 5 μm to 2 mm, or from 10 μm to 1 mm, or from 50 μm to 500 μm, or from 50 μm to 200 μm, or from 5 μm to 200 μm. When the droplet barriers have the form of solid walls, the walls can have a height equal to the gap distance between the electrowetting surface and the cover, or a height that is some fraction of the gap distance, as explained above. In various examples, the height of the solid walls can be from 5 μm to 5 mm, or from 10 μm to 2 mm, or from 50 μm to 1 mm, or from 50 μm to 500 μm, or from 5 μm to 500 μm. The solid walls can be sufficiently long to be positioned on sides of the parking electrodes to constrain droplets from drifting off the parking electrodes. As explained above, the walls may have gaps and may otherwise not extend along entire sides of the parking electrode. The gaps can include droplet entrances, exit ports for air or oil to exit when droplets move onto the parking electrode, and gaps for other purposes. These gaps can have a gap length from 5 μm to 5 mm, or from 10 μm to 3 mm, or from 50 μm to 1 mm, or from 50 μm to 500 μm, or from 5 μm to 500 μm, in some examples. The electrodes can have a width and length from 10 μm to 5 mm, or from 20 μm to 3 mm, or from 50 μm to 1 mm, or from 100 μm to 500 μm, or from 10 μm to 200 μm.

When the droplet barriers are formed as grooves, the grooves can have similar widths, lengths, and gap lengths as the solid wall barriers described above. The grooves can have any desired depth in the dielectric layer or the substrate material. In some examples, the depth of the grooves can be from 5 μm to 1 mm, or from 10 μm to 500 μm, or from 50 μm to 500 μm, or from 50 μm to 200 μm, or from 5 μm to 200 μm. The grooves can be formed by cutting into the dielectric layer material, cutting into the substrate material, or formed additively by depositing materials such as the dielectric layer material leaving an empty groove.

As used herein, “plurality of droplet barriers” can refer to droplet barriers that are positioned on multiple sides of a parking electrode. In some cases, the droplet barriers can be in the form of multiple discrete structures such as solid walls that extend up from the electrowetting surface, where the separate solid walls are not connected one to another. However, in some examples the droplet barriers can be connected together as a single solid structure. For example, three solid walls can be positioned on three sides of the parking electrode, and the three walls can connect at the corners. Although these solid walls are connected together, they can still be referred to as a “plurality” of droplet barriers. Accordingly, a variety of droplet barrier designs can be formed as a solid connected structure, but these can still be referred to as a plurality of droplet barriers because the droplet barriers encompass or constrain a plurality of sides of parking electrodes.

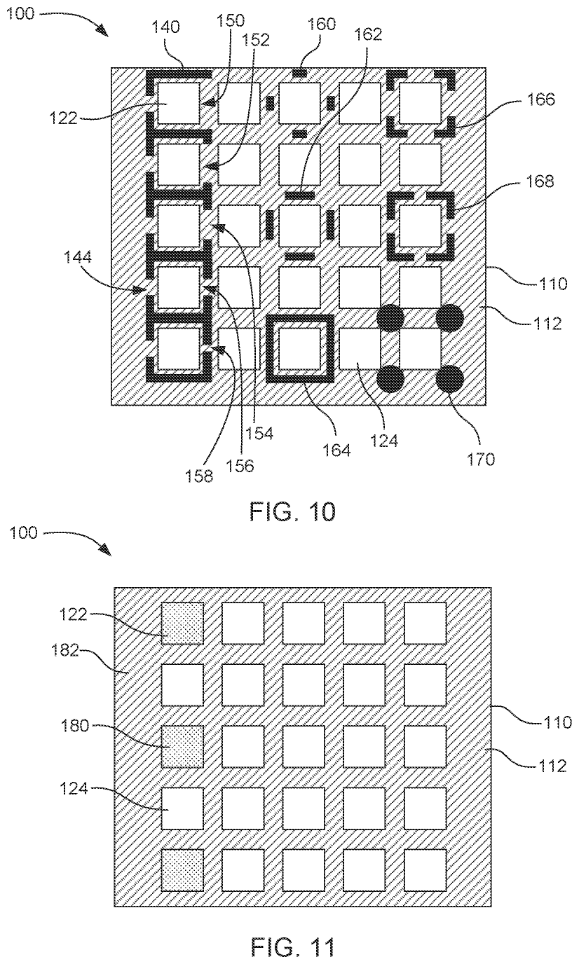

FIG. 10 illustrates an example digital microfluidic device 100 with several more example designs of droplet barriers 140. In describing the droplet barriers in this figure, the terms “right,” “left,” “top,” and “bottom” are used to describe directions from the viewpoint shown in the figure. These are used for convenience to describe the figure, and do not imply that the digital microfluidic device is oriented in any particular way. On the left edge of the electrode array is a row of parking electrodes 122. The parking electrodes along this left edge have adjacent electrodes to their right. One design includes droplet barriers with a full-width opening 150 to be used as a droplet entrance. This design also includes an exit port 144 on the left side of the parking electrode, similar to previous examples. Another design includes a large-width opening 152 to be used as a droplet entrance. Below this there are a medium-width opening 154, a narrow opening 156, and a very narrow opening 158, which are all used as droplet entrances to their respective parking electrodes. In some of these designs, the droplet entrance is wider than the exit port. In other designs, the droplet entrance is the same width as the exit port or narrower than the exit port.

FIG. 10 also shows an example in which the droplet barriers 140 include short sidewalls 160 with gaps at the corners of the parking electrode 122. Another example includes long sidewalls 162 with gaps at the corners of the parking electrode. Yet another example includes full-length sidewalls 164 that are connected at the corners. In some examples, this design can be used with walls that do not extend all the way from the electrowetting surface 110 to the cover, such as half-height walls or shorter walls. Another example shown in this figure includes short corner walls 166 positioned around the corners of the parking electrode. Another example includes long corner walls 168 positioned around the corners of the parking electrode. The final example shown in this figure includes corner columns 170 positioned at the corners of the parking electrode.

The above examples described droplet barriers that have the form of solid walls positioned on sides of the parking electrode. In alternative examples, droplet barriers can be formed by providing a surface that has greater hydrophobicity compared to the parking electrode. As explained above, the electrodes can be covered by a dielectric coating so that the liquid droplets do not actually directly contact the electrode itself. Thus, the term “greater hydrophobicity compared to the parking electrode” refers to the surface above the parking electrode that is actually in contact with the liquid droplet. By placing a more hydrophobic surface adjacent to the parking electrode, the liquid droplet can be held in place on the parking electrode because it is energetically unfavorable for the droplet to move over the more hydrophobic surface. Thus, a portion of the surface with greater hydrophobicity can act as a droplet barrier without any solid walls or other structures to physically block the droplet. However, in some examples, droplet barriers can include solid walls that have a more hydrophobic surface than the parking electrode, thus combining physical and surface energy effects to hold the droplet on the parking electrode.

The entire electrowetting surface can be hydrophobic, as mentioned above. This can be helpful for operation of the digital microfluidic device, because the hydrophobic surface helps move liquid droplets across the surface when a voltage is applied to another electrode to temporarily reduce the contact angle over that electrode. If the surface were normally hydrophilic, then the liquid droplet would tend to wet the whole surface and stick to electrodes even when no voltage is applied. Since the entire surface can be hydrophobic, the droplet barriers can be more hydrophobic than the surface over the parking electrode. This can be accomplished in several ways. In one example, the entire electrowetting surface can be made of a hydrophobic material or treated with a hydrophobic coating. Then, the parking electrode can be treated with a less-hydrophobic coating. In another example, the surface over the parking electrode can be made from a material that is less hydrophobic, and the remainder of the electrowetting surface can be made from a different material that is more hydrophobic. In these examples, a liquid droplet can be constrained and held in place over the parking electrode because the remainder of the electrowetting surface is more hydrophobic.

FIG. 11 shows an example digital microfluidic device 100 that includes an electrowetting surface 110 with regions of lower hydrophobicity 180 (shown as dot-shaded areas) over parking electrodes 122. The remainder of the electrowetting surface outside the parking electrodes is a region of greater hydrophobicity 182. Thus, the adjacent electrodes 124 that are adjacent to the parking electrodes are covered by the surface having a greater hydrophobicity. When a liquid droplet is positioned on the lower hydrophobicity surface over a parking electrode, the surrounding surfaces with greater hydrophobicity will constrain the movement of the liquid droplet and prevent the droplet from drifting if voltage is turned off to the parking electrode.

The surface with lower hydrophobicity that is positioned over the parking electrodes can have a relatively lower contact angle with water. However, the surface can still be considered hydrophobic, and thus can have a contact angle greater than 90°. In some examples, the surface over the parking electrodes can have a contact angle with water from 92° to 130°, or from 95° to 125°, or from 100° to 120°. The droplet barriers can have a surface with a higher contact angle compared to the surface over the parking electrodes. In some examples, the droplet barriers can have a contact angle from 120° to 160°, or from 125° to 155°, or from 130° to 150°, provided that the contact angle of the droplet barriers is higher than the contact angle of the parking electrodes.

In certain examples, the parking electrodes can be treated with a hydrophobic coating material, and the surface area around the parking electrodes can be treated with a super-hydrophobic coating material. For example, the parking electrode can be coated with ACULON™ ALT #6 (Aculon, USA), which has a contact angle of 102° with water, and the areas around the parking electrode can be coated with ACULON™ NC-SL2 (Aculon, USA), which has a contact angle of 140° with water.

In some examples, the parking electrodes can have a surface with lower hydrophobicity and the entire remainder of the electrowetting surface can have a higher hydrophobicity, as in the example of FIG. 11. In such examples, the entire surface around the parking electrodes acts as droplet barriers. In other examples, droplet barriers can be formed by placing more hydrophobic material on sides of the parking electrodes without covering the entire remainder of the electrowetting surface. A variety of designs for solid wall droplet barriers is described above. These designs can also be used as a pattern for hydrophobic droplet barriers. A material with greater hydrophobicity can be deposited on the electrowetting surface in the shape of these designs to form hydrophobic droplet barriers. It is also noted that hydrophobic droplet barriers can also be formed on the cover of the device. Material with a greater hydrophobicity can be placed on the inner surface of the cover in areas around the parking electrode. These areas of greater hydrophobicity on the cover can also act as droplet barriers to prevent the droplet from drifting. Any of the designs described above can also be applied to the inner surface of the cover. In further examples, the concepts of solid wall droplet barriers and hydrophobic droplet barriers can be combined by forming solid walls from a material that has a greater hydrophobicity, or forming solid walls of another material and then treating the solid walls with a more hydrophobic coating.

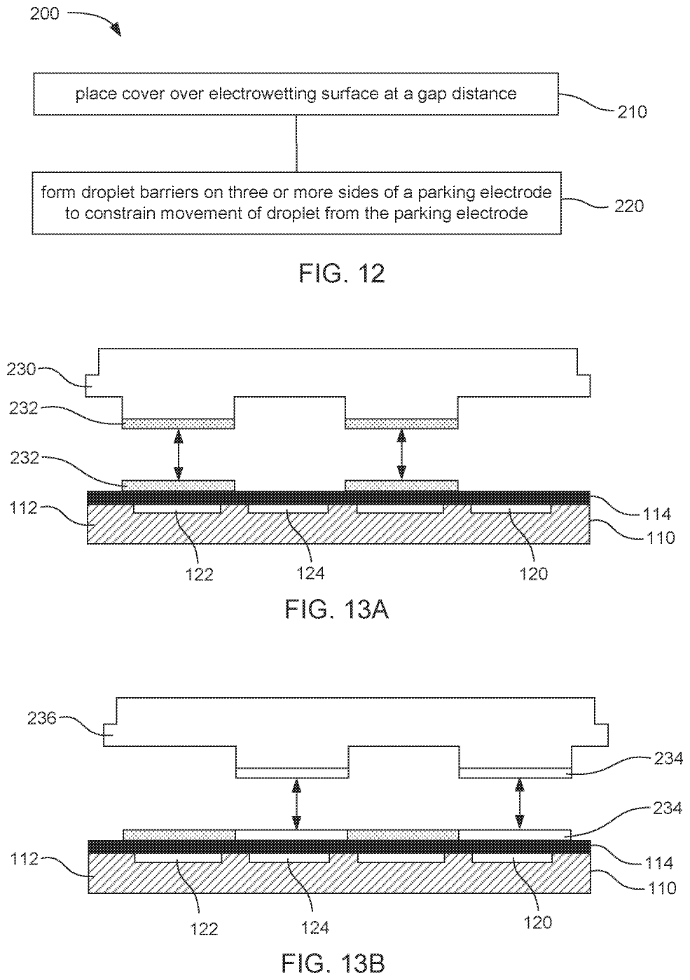

Methods of making the digital microfluidic devices described herein can depend on the type of droplet barriers used in the device. FIG. 12 is a flowchart illustrating one example method 200 of making a digital microfluidic device. This method includes: placing a cover over a hydrophobic electrowetting surface at a gap distance sufficient to accommodate a liquid droplet between the cover and the electrowetting surface, wherein the electrowetting surface includes an array of electrodes, wherein the individual electrodes have a shape with three or more sides, wherein the array of electrodes includes a parking electrode and an adjacent electrode that is adjacent to the parking electrode 210; and forming a plurality of droplet barriers positioned on three or more sides of the parking electrode, wherein the droplet barriers constrain movement of a liquid droplet from the parking electrode to the adjacent electrode 220. This method can be applied with various types of droplet barriers as described above.

In the case of droplet barriers that include solid walls, the method of making the digital microfluidic device can include forming solid walls extending from the electrowetting surface or extending from the cover. The solid walls can extend all the way across the gap distance between the cover and the electrowetting surface, or partially across the gap distance. Example methods of forming these solid walls can include molding, machining, photoresist patterning, embossing, stamping, or a combination thereof. In certain examples, the solid walls can be formed by molding the cover using a mold that includes the shape of the walls. The cover can be molded from a material such as clear plastic. Molding can be used to make features significantly smaller than 1 mm. However, if the microfluidic device is very small, such as having electrodes less than 200 μm in width, then it may be difficult to form solid wall droplet barriers by molding. In these cases, microfabrication methods can be used such as photopatterning of a photoresist. This can include using a photomask to pattern a layer of photoresist material such as SU-8 photoresist or another photopatternable material. Solid walls formed of these materials can be formed on the electrowetting surface or on the cover.

For devices that include the surface energy-type droplet barriers, the droplet barriers can be formed by forming a surface having a greater hydrophobicity compared to the parking electrode. As explained above, this means the surface that is actually contacted by the liquid droplet when the liquid droplet is positioned over the parking electrode. The parking electrode itself is not usually contacted directly by the liquid droplet because a layer of dielectric material and possibly additional layers are placed over the electrode. Example methods of forming the surface with greater hydrophobicity can include stamping a hydrophobic coating composition, selectively dispensing a hydrophobic coating composition, selective surface modification using a stencil, forming a hydrophobic nanostructure, laser etching, reactive ion etching, or a combination thereof.

FIGS. 13A-13B show one example method of making surface energy-type droplet barriers. FIG. 13A shows a side view of an electrowetting surface 110 including with a substrate 112, an array of electrodes 120, and a dielectric layer 114. A stamp 230 is used to stamp a lower-hydrophobicity coating composition 232 over the parking electrodes 122. FIG. 13B shows a second stamp 236 being used to stamp a higher-hydrophobicity coating composition 234 onto the adjacent electrodes 124. The higher-hydrophobicity coating composition forms a coating that is more hydrophobic than the lower-hydrophobicity coating composition. The areas where the higher-hydrophobicity coating composition is stamped can act as droplet barriers around the parking electrodes.

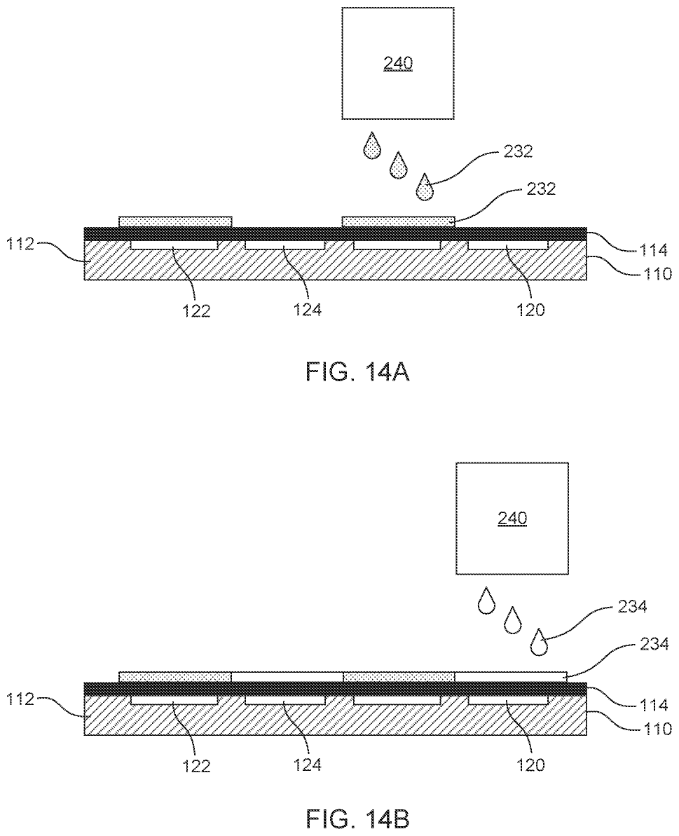

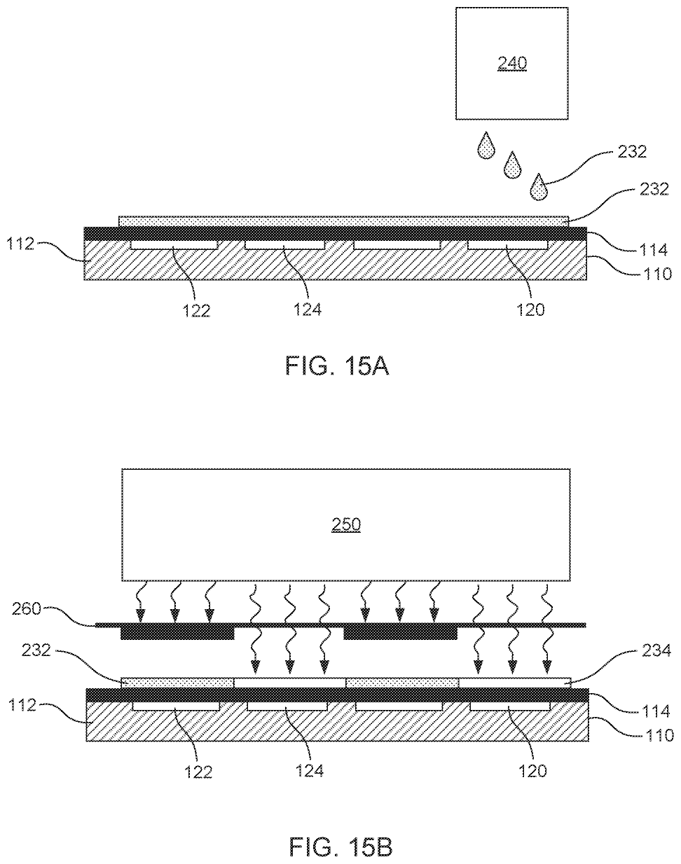

FIGS. 14A-14B show another example method of making surface energy-type droplet barriers. FIG. 14A shows a side view of an electrowetting surface 110 including a substrate 112, an array of electrodes 120, and a dielectric layer 114. A fluid dispenser 240 is used to selectively dispense a lower-hydrophobicity coating composition 232 over the parking electrodes 122. The fluid dispenser can be any suitable type of fluid dispensing device, such as an inkjet printer. FIG. 14B shows a fluid dispenser being used to dispense a higher-hydrophobicity coating composition 234 onto the adjacent electrodes 124.

FIGS. 15A-15B show another example method of making surface energy-type droplet barriers. FIG. 15A shows another example electrowetting surface 110 including a substrate 112, an array of electrodes 120, and a dielectric layer 114. A fluid dispenser 240 is used to apply a lower-hydrophobicity coating composition 232 on the entire electrowetting surface. Thus, the entire surface has a lower-hydrophobicity coating to begin with. FIG. 15B shows a treatment head 250 applying a surface treatment such as a plasma treatment or a laser treatment to the coating. The treatment modifies the surface so that the surface is more hydrophobic. A stencil 260 is placed over the surface. The stencil covers the parking electrodes and has openings for the adjacent electrodes so that the coating over the parking electrodes maintains its lower hydrophobicity. The adjacent electrodes have a higher hydrophobicity after the surface treatment. Thus, the more hydrophobic surfaces over the adjacent electrodes can act as droplet barriers around the parking electrodes.

In more detail regarding the structure of the digital microfluidic devices, the devices can include a dielectric layer over the array of electrodes as mentioned above. In some examples, the dielectric layer can have a thickness from 100 μm to 3 mm. In further examples, the thickness can be from 100 μm to 2 mm, or from 100 μm to 1 mm, or from 100 μm to 500 μm, or from 500 μm to 3 mm, or from 500 μm to 2 mm, or from 500 μm to 1 mm.

A variety of materials can be used to form the dielectric layer. In some examples, the dielectric material can include a polymer such as polydimethylsiloxane, epoxy, fluoroalkylsilane, silicone, polyolefin, polysilazane, polyvinylfluoride, polyvinylidene fluoride, polytetrafluoroethylene, polychlorotrifluoroethylene, perfluoroalkoxy polymer, fluorinated ethylene-propylene, polyethylenetetrafluoroethylene, polyethylenechlorotrifluoroethylene, perfluorinated elastomer, tetrafluoroethylene-propylene, perfluoropolyether, perfluorosulfonic acid, B-staged bisbenzocyclobutene, polybenzoxazole, parylene, or a combination thereof. Inorganic materials can also be included, such as alumina, silica, aluminum nitride, or a combination thereof. In some examples, the dielectric material can include a polyimide material such as a KAPTON® material obtainable from DuPont de Nemours, Inc. (USA) or UPILEX® films from UBE Industries (Japan). In further examples, the dielectric material can include a polyetherimide (PEI) material. In certain examples, the dielectric layer can have a thickness from 100 nm to 1 mm or from 100 nm to 100 μm, or from 100 nm to 25 μm, in some examples

In some examples, the dielectric material can have a dielectric strength of 50 V/μm to 500 V/μm, while in some examples, the dielectric strength may be from 100 V/μm to 500 V/μm. In some examples, the dielectric strength can be from 200 V/μm to 400 V/μm. In some examples, the dielectric strength can be from 300 V/μm to 500 V/μm.

A cover can be positioned over the dielectric layer at a gap distance from the dielectric layer. In some examples, the cover can be electrically grounded. The difference in charge between the charge applied to the electrodes and the cover can provide an electric field within the gap that moves droplets across the electrowetting surface. In some examples, the cover can include a conductive layer. The conductive layer can be made of a transparent conductive material in some cases, such as indium tin oxide or zinc tin oxide.

It is noted that the above figures may not be drawn to scale. In practice, the electrowetting surface and cover can have a much smaller thickness compared to their width and length. Additionally, the electrode arrays shown in the figures include a small number of electrodes for the sake of simplicity. However, in practice the electrode array is likely to include many more electrodes, such as an array having from 20 to 10,000 electrodes in some examples. The electrode array can also have any shape and arrangement of electrodes, not limited to simple square or rectangular arrays. Any arrangement of multiple electrodes can be used if the electrodes are spaced closely enough for liquid droplets to move between the electrodes by electrowetting forces. It is also noted that in some examples, additional layers of materials can be added that were not shown in the figures. As an example, in some cases adhesive layers can be used between some of the material layers shown in the figures. Accordingly, the various layers are not limited or constrained by having certain layers in direct contact with other layers as shown in the figures. In some examples, additional layers may be placed between layers that are shown in direct contact in the figures.

Regarding the surfaces in the digital microfluidic devices that come in contact with the liquid droplets, it can be useful to use hydrophobic surfaces. Aqueous liquid droplets can have a high contact angle on hydrophobic surfaces. However, applying a sufficient electric field across the gap between the electrowetting surface and the cover can reduce the contact angle of the liquid droplet. The liquid droplet can be moved by applying an electric field adjacent to the location of the liquid droplet, and this can cause the liquid droplet to move into the area of the electric field where the contact angle is lower. In some examples, a hydrophobic monolayer coating can be applied on the surfaces inside the gap. Examples of such hydrophobic monolayer coatings include FLUOROPEL™ hydrophobic coatings, available from CYTONIX (USA); RAIN-X® coatings, available from ITW Global Brands (USA); AQUAPEL™ coatings, available from PGW Auto Glass, LLC (USA); octadecyltrichlorosilane; dodecyltrichlorosilane; and others. Other types of hydrophobic surfaces can include a layer of a bulk hydrophobic material, such as a bulk polymer or a bulk ceramic material. The terms “bulk polymer” and “bulk ceramic” refer to a thicker layer of a solid homogeneous material, as opposed to a monolayer coating. Some examples of bulk polymers include TEFLON™ AF 1600 and AF 2400, available from The Chemours Company (USA); CYTOP® fluoropolymer, available from AGC chemicals Company (USA); NOVEC™ 1700 available from 3M (USA); and others. Examples of bulk ceramic materials include silicon oxycarbide, cerium oxide, and others. Other examples of hydrophobic surfaces include nanoceramic coatings. Nanoceramic coatings can include ceramic nanoparticles bound together by a polymeric binder. As used herein, “nanoparticles” can refer to particles that are from 1 nm to 1,000 nm in size. In particular examples, the nanoceramic nanoparticles used in the coating can have an average particle size from 1 nm to 200 nm, or from 5 nm to 100 nm, or from 10 nm to 60 nm, or from 60 nm to 150 nm.

As used herein, “average particle size” refers to a number average of the diameter of the particles for spherical particles, or a number average of the volume equivalent sphere diameter for non-spherical particles. The volume equivalent sphere diameter is the diameter of a sphere having the same volume as the particle. Average particle size can be measured using a particle analyzer such as the MASTERSIZER™ 3000 available from Malvern Panalytical (United Kingdom). The particle analyzer can measure particle size using laser diffraction. A laser beam can pass through a sample of particles and the angular variation in intensity of light scattered by the particles can be measured. Larger particles scatter light at smaller angles, while smaller particles scatter light at larger angles. The particle analyzer can then analyze the angular scattering data to calculate the size of the particles using the Mie theory of light scattering. The particle size can be reported as a volume equivalent sphere diameter.

The gap between the electrowetting surface and the cover can be sufficient to accommodate liquid droplets over the electrodes of the electrode array. In some examples, the gap distance can be from 50 μm to 500 μm, from 100 μm to 150 μm, or from 150 μm to 250 μm. In further examples, liquid droplets in the gap can have a droplet volume from 10 pL to 30 μL. Liquid droplets in the gap can be surrounded by air in some examples, while in other examples the gap can be filled with a dielectric oil and the liquid droplets can be an aqueous liquid that does not mix with the dielectric oil. In some examples, the dielectric oil can affect electrowetting forces on the aqueous liquid droplets, and/or resist evaporation of the aqueous liquid droplets, and/or facilitate sliding of the droplets and maintaining droplet integrity. Oils that can be used to fill the gap include silicone oil, fluorocarbon oil, engineered fluids, and others. Some specific examples can include 2 centistoke silicone oil, 5 centistoke silicone oil, FLUOROINERT™ FC-40 and FC-75 available from Sigma Aldrich (USA), NOVEC™ HFE 7100, HFE 7300, and HFE 7500 available from 3M (USA).

It is noted that many of the examples described above include aqueous liquid droplets on the electrowetting surface. The electrowetting effect can be particularly useful with aqueous liquids, especially with aqueous liquids that include electrolytes. However, non-aqueous liquids can also be manipulated on the electrowetting surface. Some examples of non-aqueous liquids that can be manipulated with the electrowetting surface include formamide, formic acid, dimethyl sulfoxide, N,N-dimethylformamide, acetonitrile, methanol, ethanol, acetone, piperidine, 1-pentanol, 1-hexanol, dichloromethane, dibromomethane, tetrahydrofuran, m-dichlorobenzene, chloroform, 4-methyl-3-heptanol, and others. Some non-aqueous fluids may move across the electrowetting surface when a more intense electric field is used, such as using a higher voltage or smaller gap distance between the top electrodes and bottom electrodes. In other examples, aqueous liquids can be moved using a less intense electric field. In certain examples, the voltage applied to the electrodes can be from 100 V to 400 V, or from 200 V to 400 V, or from 200 V to 300 V.

Regarding the electrodes of the electrode array, the electrodes can be formed of a conductive material, such as metal, a conductive ceramic, or other conductive materials. Some specific examples can include copper, copper plated with gold, gold, platinum, silver, aluminum, graphene, graphitic materials, indium tin oxide, zinc tin oxide, and others. In some examples, the electrode array can also include conductive traces that lead to the individual electrodes, and the conductive traces can be connectable to a power source and/or an electronic controller to allow individual electrodes to be powered. In some examples, the conductive electrodes and traces can be deposited using a suitable deposition process, such as physical vapor deposition, chemical vapor deposition, electroplating, electroless plating, conductive ink printing, photo-etching, or combinations thereof. The thickness of the electrodes can be from 50 nm to 100 μm, or from 100 nm to 10 μm, or from 100 nm to 1 μm, in some examples.

Digital microfluidic devices can also include a controller electrically connected or connectable to the array of electrodes. The controller can be configured to selectively apply charge to certain electrodes to create an electric field at the electrowetting surface in order to control liquid droplets as explained above. The controller can be configured to perform operations with liquid droplets, such as moving droplets, splitting droplets, merging droplets, and others. In some examples, the controller can be referred to as being programmed to perform the operations explained above. In certain examples, the controller can include a processor in electronic communication with a memory. The processor can execute instructions stored in the memory, and these instructions can cause the controller to perform the operations. In particular, the controller can generate and transmit electric signals to the electrode array, and any other electronic components in the digital microfluidic device, so that the digital microfluidic device manipulates liquid droplets in a desired way. In some examples, the controller can be a general-purpose computing device connected to the other electronic components of the digital microfluidic device. In other examples, the controller can be a dedicated controller incorporated in a digital microfluidic device. In still further examples, a combination of a dedicated controller and a separate general-purpose computing device can be used to control the digital microfluidic device. For example, a general-purpose computing device can be used to provide a user interface to allow a user to input commands, while a dedicated controller integrated in the digital microfluidic device can interpret the commands and generate signals to individual electrodes in the electrode array.

The memory can include random access memory (RAM), read only memory (ROM), a mass storage device, or another type of storage that includes a non-transitory tangible medium or non-volatile tangible medium. The memory can store machine readable instructions that can cause the controller to perform the operations described above.

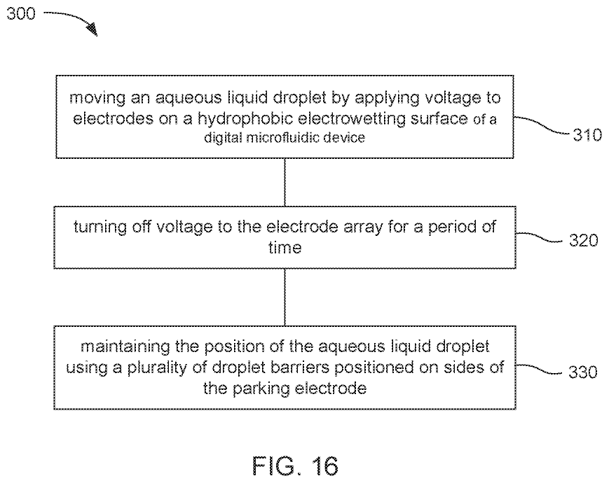

In various examples, methods of processing liquids using the digital microfluidic devices described herein can include using the devices to perform any of the operations described above. In particular, the methods can include moving a liquid droplet onto a parking electrode and then maintaining the droplet over the parking electrode for a period of time. FIG. 16 is a flowchart illustrating one example method 300 of processing a liquid in a digital microfluidic device. This method includes moving 310 an aqueous liquid droplet by applying voltage to electrodes on a hydrophobic electrowetting surface of a digital microfluidic device, wherein the electrowetting surface includes an array of electrodes, wherein the array of electrodes includes a parking electrode and an adjacent electrode that is adjacent to the parking electrode. Furthermore, while the aqueous liquid droplet is positioned over the parking electrode, the method includes turning off 320 voltage to the electrode array for a period of time. The method also includes maintaining 330 the position of the aqueous liquid droplet over the parking electrode during the period of time using a plurality of droplet barriers positioned on sides of the parking electrode. The droplet barriers can have any of the characteristics and shapes described above.

The voltage can be turned off to the electrode array or to the entire digital microfluidic device for a period of time. During this time, the device can be stored or packed and shipped. The droplet barriers can hold the droplet in place over the parking electrode during storage or shipping. In other examples, the digital microfluidic device can be heated. Applying heat can be useful for incubation or thermal cycling, which may be useful in DNA amplification, growth of cells, and in other applications. In some cases, the digital microfluidic device can be loaded into an oven for heating while the voltage is disconnected. In further examples, the voltage can be disconnected in order to load the digital microfluidic device into another instrument for data analysis. The instrument can include a microscope, chromatograph, fluorometer, or a variety of other instruments.

### Definitions

It is to be understood that this disclosure is not limited to the particular processes and materials disclosed herein because such processes and materials may vary somewhat. It is also to be understood that the terminology used herein is used for the purpose of describing particular examples. The terms are not intended to be limiting because the scope of the present disclosure is intended to be limited by the appended claims and equivalents thereof.

It is noted that, as used in this specification and the appended claims, the singular forms “a,” “an,” and “the” include plural referents unless the context clearly dictates otherwise.

As used herein, the term “substantial” or “substantially” when used in reference to a quantity or amount of a material, or a specific characteristic thereof, refers to an amount that is sufficient to provide an effect that the material or characteristic was intended to provide. The exact degree of deviation allowable may in some cases depend on the specific context.

As used herein, the term “about” is used to provide flexibility to a numerical range endpoint by providing that a given value may be “a little above” or “a little below” the endpoint. The degree of flexibility of this term can be dictated by the particular variable and determined based on the associated description herein.

As used herein, a plurality of items, structural elements, compositional elements, and/or materials may be presented in a common list for convenience. However, these lists should be construed as though the members of the list are individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include the numerical values explicitly recited as the limits of the range, and also to include individual numerical values or sub-ranges encompassed within that range as if the numerical values and sub-ranges are explicitly recited. As an illustration, a numerical range of “about 1 wt % to about 5 wt %” should be interpreted to include the explicitly recited values of about 1 wt % to about 5 wt %, and also to include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting a single numerical value. Furthermore, such an interpretation should apply regardless of the breadth of the range or the characteristics being described.

### EXAMPLES

The following prophetic examples illustrate example digital microfluidic devices according to the present disclosure. However, it is to be understood that the following are merely illustrative of the application of the principles of the present disclosure. Numerous modifications and alternative devices, methods, and systems may be devised without departing from the scope of the present disclosure.

### Example 1—Solid Wall Droplet Barriers

A digital microfluidic device is constructed with the design shown in FIG. 1. The device includes droplet barriers formed as solid walls that are molded as a part of the cover of the device. The solid walls extend down from the cover to the electrowetting surface. A droplet entrance opening is left open on one side of the parking electrode, and an exit port is left open on the opposite side of the parking electrode. The gap volume between the cover and the electrowetting surface is filled with silicon oil, and a droplet of an aqueous liquid is added. The droplet is moved across the electrode array using electrowetting forces by applying voltages to selected electrodes. The droplet is then moved from the adjacent electrode onto the parking electrode. At this point, the digital microfluidic device is disconnected from power so that no voltage is applied to any of the electrodes. The solid wall droplet barriers prevent the liquid droplet from drifting off the parking electrode, and the droplet remains on the parking electrode for a desired period of time.

### Example 2—Surface Energy-Type Droplet Barriers

A digital microfluidic device is constructed having the design shown in FIG. 11. The device is made by preparing an electrowetting surface having a substrate, an electrode array on the substrate, and a dielectric layer over the electrode array and the substrate. A stamp is used to apply a lower-hydrophobicity coating composition (ACULON™ ALT #6 from Aculon, USA) over the parking electrodes. Another stamp is used to apply a higher-hydrophobicity coating composition (ACULON™ NC-SL2 from Aculon, USA) to the rest of the electrowetting surface. The gap volume between the cover and the electrowetting surface is filled with silicon oil, and a droplet of an aqueous liquid is added. The droplet is moved across the electrode array using electrowetting forces by applying voltages to selected electrodes. The droplet is then moved onto one of the parking electrodes from an adjacent electrode. The digital microfluidic device is disconnected from power. The higher hydrophobicity coating around the parking electrode acts as droplet barriers on the sides of the parking electrode, which prevent the liquid droplet from drifting off the parking electrode.

While the present technology has been described with reference to certain examples, various modifications, changes, omissions, and substitutions can be made without departing from the disclosure.

What is claimed is:

1. A digital microfluidic device, comprising:
a hydrophobic electrowetting surface comprising an array of electrodes, wherein the individual electrodes have a shape with three or more sides, wherein the array of electrodes includes a parking electrode and an adjacent electrode that is adjacent to the parking electrode;
a cover positioned over the electrowetting surface at a gap distance sufficient to accommodate a liquid droplet between the cover and the electrowetting surface; and
a plurality of droplet barriers positioned on three or more sides of the parking electrode, wherein the droplet barriers constrain movement of a liquid droplet from the parking electrode to the adjacent electrode.

2. The digital microfluidic device of claim 1, wherein the droplet barriers comprise a surface having a greater hydrophobicity compared to the parking electrode.

3. The digital microfluidic device of claim 1, wherein the droplet barriers comprise solid walls occupying a space between the cover and the electrowetting surface.

4. The digital microfluidic device of claim 3, wherein the solid walls extend across the entire gap distance between the electrowetting surface and the cover.

5. The digital microfluidic device of claim 3, wherein the solid walls extend partially across the distance between the electrowetting surface and the cover.

6. The digital microfluidic device of claim 3, wherein the solid walls are formed as a part of the electrowetting surface or as a part of the cover.

7. The digital microfluidic device of claim 1, wherein the droplet barriers comprise grooves formed in the electrowetting surface.

8. The digital microfluidic device of claim 1, further comprising an oil filling a volume between the cover and the electrowetting surface.

9. The digital microfluidic device of claim 8, further comprising a droplet of an aqueous liquid positioned over the parking electrode.

10. A method of making a digital microfluidic device, comprising:
placing a cover over a hydrophobic electrowetting surface at a gap distance sufficient to accommodate a liquid droplet between the cover and the electrowetting surface, wherein the electrowetting surface comprises an array of electrodes, wherein the individual electrodes have a shape with three or more sides, wherein the array of electrodes includes a parking electrode and an adjacent electrode that is adjacent to the parking electrode; and
forming a plurality of droplet barriers positioned on three or more sides of the parking electrode, wherein the droplet barriers constrain movement of a liquid droplet from the parking electrode to the adjacent electrode.

11. The method of claim 10, wherein forming the plurality of droplet barriers comprises forming solid walls extending from the electrowetting surface or extending from the cover, wherein the solid walls are formed by molding, machining, photoresist patterning, embossing, stamping, and a combination thereof.

12. The method of claim 10, wherein forming the plurality of droplet barriers comprises forming a surface having a greater hydrophobicity compared to the parking electrode, wherein the surface having a greater hydrophobicity is formed by stamping a hydrophobic coating composition, selectively dispensing a hydrophobic coating composition, selective surface modification using a stencil, forming a hydrophobic nanostructure, laser etching, reactive ion etching, or a combination thereof.

13. A method of processing a liquid in a digital microfluidic device, comprising:
moving an aqueous liquid droplet by applying voltage to electrodes on a hydrophobic electrowetting surface of a digital microfluidic device, wherein the electrowetting surface comprises an array of electrodes, wherein the array of electrodes includes a parking electrode and an adjacent electrode that is adjacent to the parking electrode;
while the aqueous liquid droplet is positioned over the parking electrode, turning off voltage to the electrode array for a period of time; and
maintaining the position of the aqueous liquid droplet over the parking electrode during the period of time using a plurality of droplet barriers positioned on sides of the parking electrode.

14. The method of claim 13, further comprising, during the period of time while the voltage is turned off, storing the digital microfluidic device, shipping the digital microfluidic device, loading the digital microfluidic device into an oven, applying heat to the digital microfluidic device, applying thermal cycling to the digital microfluidic device, loading the digital microfluidic device into an instrument for data analysis, or a combination thereof.

15. The method of claim 13, wherein the digital microfluidic device comprises a cover positioned over the electrowetting surface at a gap distance sufficient to accommodate the aqueous liquid droplet between the cover and the electrowetting surface, and wherein the droplet barriers comprise a surface having a greater hydrophobicity compared to the parking electrode, or solid walls occupying a space between the cover and the electrowetting surface, or a combination thereof.
